SEQUENCE LISTING (14 sequences)

SEQ ID NO: 1 - protein, 331 aa, synthetic construct
  REGION 1..331: note = ARG-1 with histidine tag
MGHHHHHHGSSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 2 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 3 - protein, 30 aa, synthetic construct
  REGION 1..30: note = B-chain
FVNQHLCGSHLVEALYLVCGERGFFYTPKT

SEQ ID NO: 4 - protein, 35 aa, synthetic construct
  REGION 1..35: note = C-peptide
RREAEDLQVGQVELGGGPGAGSLQPLALEGSLQKR

SEQ ID NO: 5 - protein, 21 aa, synthetic construct
  REGION 1..21: note = A-chain / polypeptide (ii)
GIVEQCCTSICSLYQLENYCN

SEQ ID NO: 6 - protein, 50 aa, synthetic construct
  REGION 1..50: note = (G4S)10 linker
GGGGSGGGGSGGGGSGGGGSGGGGSGGGGSGGGGSGGGGSGGGGSGGGGS

SEQ ID NO: 7 - protein, 417 aa, synthetic construct
  REGION 1..417: note = fusion protein
  SITE 9: note = X = linker, particularly a GS linker
  SITE 331: note = X = linker, particularly a (GGGGS)n linker, wherein n is from 1 to 10
MGHHHHHHXSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPKXFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 8 - protein, 361 aa, synthetic construct
  REGION 1..361: note = polypeptide (i)
  SITE 9: note = X = linker, particularly a GS linker
  SITE 331: note = X = linker, particularly a (GGGGS)n linker, wherein n is from 1 to 10
MGHHHHHHXSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPKXFVNQHLCGSHLVEALYLVCGERGFFYTPKT

SEQ ID NO: 9 - protein, 5 aa, synthetic construct
  REGION 1..5: note = G4S linker
GGGGS

SEQ ID NO: 10 - protein, 9 aa, synthetic construct
  REGION 1..9: note = His-Tag
GHHHHHHGS

SEQ ID NO: 11 - protein, 30 aa, synthetic construct
  REGION 1..30: note = B-chain
FVNQHLCGSDLVEALYLVCGERGFFYTDPT

SEQ ID NO: 12 - protein, 6 aa, synthetic construct
  REGION 1..6: note = C-linker
GGGPRR

SEQ ID NO: 13 - protein, 21 aa, synthetic construct
  REGION 1..21: note = A-chain
GIVEQCCHSICSLYQLENYCN

SEQ ID NO: 14 - protein, 57 aa, synthetic construct
  REGION 1..57: note = SCI-57
FVNQHLCGSDLVEALYLVCGERGFFYTDPTGGGPRRGIVEQCCHSICSLYQLENYCN

(19) United States
(12) Patent Application Publication
(10) Pub. No.: US 2025/0376671 A1
(43) Pub. Date: Dec. 11, 2025

(54) ARGINASE-INSULIN FUSION PROTEIN

(71) Applicant: Kyon Biotech AG, Zürich (CH)

(72) Inventor: Slobodan TEPIC, Klosters Dorf (CH)

(73) Assignee: Kyon Biotech AG, Zürich (CH)

(21) Appl. No.: 18/693,489

(22) PCT Filed: Mar. 23, 2023
(86) PCT No.: PCT/EP2022/075887

§ 371 Date: Mar. 19, 2024

(30) Foreign Application Priority Data
Sep. 20, 2021 (EP) 21197781.4

(51) Int. Cl. C12N 9/78; A61K 31/7004; A61K 38/00; A61P 35/00; C07K 14/62

(52) U.S. Cl. CPC C12N 9/78; A61K 31/7004; A61P 35/00; C07K 14/62; A61K 38/00; C07K 2319/21; C12Y 305/03001

(57) ABSTRACT

The invention discloses a fusion protein of insulin and arginase useful as an anti-tumor medication, an anti-obesity medication or a type-2 diabetes medication.

### CROSS REFERENCE TO RELATED APPLICATION(S)

This application is a 35 U.S.C. § 371 National Stage of International Patent Application No. PCT/EP2022/075887, filed Sep. 19, 2022, which claims priority to and the benefit of European Patent Application No. 21197781.4, filed Sep. 20, 2021, the disclosure of which is incorporated herein by reference in its entirety.

The present invention relates to fusion proteins of arginase and insulin and their use in medicine, particularly for the treatment of cancer and metabolic disorders such as obesity or diabetes, e.g., diabetes type 2.

### SEQUENCE LISTING

The present application includes a Sequence Listing filed in ST.26 format. The Sequence Listing, created on Feb. 7, 2025, is entitled “2923-1447-SEQ-Listing.xml” and is 20,985 bytes in size. The information in the electronic format of the Sequence Listing is part of the present application and is incorporated herein by reference in its entirety.

### BACKGROUND

Arginine depletion has been shown of utility in treating some cancers, such as hepatocellular carcinoma and melanoma, and based on in vitro work, probably many others. The use of arginine depleting enzymes such as arginase in cancer therapy has e.g., been described by Shen et al. (Cell Death & Disease 8 (2017), e2720), Zou et al. (Biomedicine & Pharmacotherapy 118 (2019), 109210), Al-Koussa et al. (Cancer Cell International 20 (2020) Article number 150) and Zhang et al. (Cancer Letters 502 (2012), 58-70), the contents of which are herein incorporated by reference.

Use of arginine converting enzymes is necessary, but our own research has shown it not being sufficient to cause and maintain systemic, deep depletion of arginine needed to cause rapid, selective killing of cancer cells.

Use of an insulin/glucose clamp in parallel with enzymatic degradation of arginine makes the task of deep arginine depletion much more manageable. Insulin is a growth factor and thus promotes protein synthesis and inhibits protein breakdown. This is of crucial importance when the task is removal from circulation of any amino acid, and particularly of arginine, which is a semi-essential amino acid under tight homeostatic control.

Increase of vascular permeability by insulin also helps in getting therapeutic enzymes into interstitial fluid space, closer to where most cancerous cells reside.

And finally, insulin may also play a role in transporting arginine degrading enzymes into cancerous cells by stimulating endocytosis. For this to work, an enzyme molecule needs to be in close proximity at the time the insulin molecule attaches to the insulin receptor, which is subject to chance and concentration of the enzyme.

It was an object of the present invention, to overcome disadvantages associated with previous treatment schedules involving amino acid depletion, e.g., arginine depletion.

### DESCRIPTION OF THE INVENTION

A first aspect of the present invention relates to a fusion protein comprising a first domain and a second domain wherein the first domain comprises an amino acid degrading enzyme and the second domain comprises an insulin.

A further aspect of the invention relates to a nucleic acid molecule encoding the fusion protein.

A further aspect of the invention relates to a host cell transfected with the nucleic acid molecule.

A further aspect of the invention relates to a method of producing the fusion protein by cultivating the host cell and obtaining the fusion protein from the host cell or from the culture medium.

A further aspect of the invention relates to the fusion protein for use in medicine.

In certain embodiments, the first domain (enzyme) is located N-terminally to the second domain (insulin). In further embodiments, the second domain (insulin) is located N-terminally to the first domain (enzyme).

In certain embodiments, the fusion protein is a genetic fusion, which may be produced in a recombinant host cell by expression of a nucleic acid molecule, particularly a DNA molecule encoding the fusion protein or a precursor thereof, and optional subsequent processing.

In certain embodiments, the fusion protein is a non-genetic fusion wherein the first domain and the second domain are produced separately, e.g., in a recombinant host cell, and subsequently linked with each other, e.g., by covalent bonds.

The first domain of the fusion protein comprises an amino acid degrading enzyme. In certain embodiments, the amino acid degrading enzyme is an arginine degrading enzyme, e.g., an arginine deiminase (ADI; EC 3.5.3.6; UniProt-P23793) or an arginase. In particular embodiments, the amino acid degrading enzyme is human liver arginase (human Arginase-1; ARG1; EC 3.5.3.1; Uni-Prot-P05089), or human kidney arginase (human Arginase-2; ARG2; EC 3.5.3.1; Uni-Prot-P78540).

A modification of human liver arginase (ARG1) or human kidney arginase (ARG2) to replace manganese with cobalt and to shift the optimum pH to that of plasma is also particularly suitable for fusion with insulin according to the present invention. A Co²⁺ modified recombinant human arginase I is described by Stone E M, Glazer E S, Chantranupong L, et al. (Replacing Mn(2+) with Co(2+) in human arginase enhances cytotoxicity toward L-arginine auxotrophic cancer cell lines, ACS Chem Biol. 2010;5 (3): 333-342, doi: 10.1021/cb900267j) and in US 20121/0189371 A1, the contents of which are herein incorporated by reference.

Several other amino acids have been targeted for cancer treatment, e.g., tryptophan by tryptophan dioxygenase (TDO2; EC 1.13.11.11, UniProt-P48775) or methionine by S-adenosylmethionine synthase (MAT1A; EC 2.5.1.6; UniProt-Q00266). However, arginine depletion is considered the most effective approach to cancer treatment.

Since the early seventies, asparaginase has been the most successfully used enzymatic treatment for cancers, particularly for childhood acute lymphoblastic leukemia (ALL). Asparaginase is active only in its tetrameric form, which at approximately 130 kDa is too large to be used as such. According to the present invention, it is delivered in a dissociated form, e.g., dissolved in urea in its monomeric form as described in WO 2020/245041, the content of which is herein incorporated by reference, wherein each of the monomers is fused with insulin. In such case, extravasation is possible and followed by reconstitution into tetramer in the interstitial fluid it can yield an active form of the enzyme. Thus, asparaginase is also a preferred enzyme to be used in this invention.

In certain embodiments, the amino acid degrading enzyme is a monomeric protein, e.g., a monomeric arginase.

The second domain of the fusion protein comprises an insulin including a precursor thereof such as a proinsulin, from which an insulin may be obtained by enzymatic cleavage including self-cleavage.

In certain embodiments, the insulin is a human insulin or an insulin analogue, e.g., a fast-acting insulin such as insulin glulisine, insulin aspart, insulin lispro, or a slow-acting insulin such as insulin NPH, insulin glargine, insulin detemir or insulin degludec. These insulins typically comprise an A-chain and a B-chain linked by S-S bridges and may be obtained by cleavage from a corresponding proinsulin.

In certain embodiments, the fusion protein of the invention is produced as a precursor wherein the first domain comprises a proinsulin which is subsequently cleaved to the corresponding insulin, e.g., by autocatalysis.

Alternatively, the insulin may be a single-chain insulin, e.g., an insulin or insulin analogue wherein an insulin B-chain and an insulin A-chain, which optionally contain at least one amino acid modification, are connected by a permanent linker. Single-chain insulins are, e.g., described by Glidden et al. (J. Biol. Chem. 293 (2018), 47-68, or Mao et al. (Appl. Microbiol. Biotechnol. 103 (2019), 8737-8751) the contents of which are herein incorporated by reference. Single-chain insulins are also described in patents U.S. Pat. No. 8,192,957; 8,501,440; 8,921,313; 15 8,993,516; 9,079,975; 9,200,053; 9,388,228; 9,499,600; 9,624,287; 9,758,563; 9,975,940; 10,392,429; 10,472,406; and 10,822,386, the contents of which are herein incorporated by reference. In a particular embodiment, the single-chain insulin is SCI-57 comprising a permanent hexapeptide linker GGGPRR (SEQ ID NO. 12) between the B and A-chains as described in Hua QX, Nakagawa SH, Jia W, et al., Design of an active ultrastable single-chain insulin analog: synthesis, structure, and therapeutic implications. J Biol Chem. 2008;283 (21): 14703-14716. doi: 10.1074/jbc.M800313200, the content of which is herein incorporated by reference.

In certain embodiments, the first domain and the second domain are directly connected to each other. In further embodiments, the first domain and the second domain are connected to each other by a linker, e.g., a linker comprising 1-100, particularly 10-60 amino acids

The linker may be a flexible linker, e.g., a linker composed of the amino acids G and S, e.g., a (GmS) n linker wherein m is from 1-5 and n is from 1-10. Alternatively, the linker may be rigid linker, e.g., comprising at least one P residue. In certain embodiments, the linker may be a cleavable linker, e.g., comprising a proteolytic cleavage site.

In certain embodiments, the fusion protein may comprise additional domains, including a purification domain such as a His-tag, a FLAG domain etc., a secretion domain or another functional domain.

In certain embodiments, the fusion protein may be conjugated to a heterologous, e.g., non-proteinaceous moiety such as polyethylene glycol (PEG) or to a heterologous protein to extend its plasma half-life. In such a case, it will be advantageous to select a small conjugation partner thereby still allowing extravasation. In preferred embodiments, the fusion protein has a molecular mass lower than about 70 kDa, e.g., 60 kDa or lower. In further preferred embodiments, the fusion protein is unPEGylated.

The fusion protein of the invention is useful in medicine including veterinary and human medicine, for example as a medication in the treatment of cancers, e.g., leukemias, lymphomas, hepatocellular carcinoma, melanoma, colon carcinoma, osteosarcoma, soft tissue sarcomas, mast cell tumors, or in the prevention or treatment of a metabolic disorder, e.g., obesity or diabetes, particularly type 2 diabetes.

The fusion protein of the invention is typically administered by injection or infusion. In particular embodiments, administration is accompanied by co-administration of glucose including administration of a glucose-providing oligo-or polysaccharide such as maltose, dextrin, starch etc., in order to maintain a sufficient glucose level of e.g., about 4.0 to about 10 mM. Further, administration of the fusion protein may be accompanied by certain measures to compensate side-effects of arginine depletion such as infusion of a nitric oxide (NO) donor, e.g., sodium nitroprusside (SNP), and/or a pressor peptide, e.g., a vasopressin, to balance NO-induced vasodilation. Arginine is the only precursor for synthesis of short-lived NO. All pressor peptides contain arginine and are short-lived. Co-infusion of Iloprost, a prostacyclin analog has also been found useful in the maintenance of thrombocytes.

The fusion protein may be administered as a monotherapy or in combination with further active agents, e.g., anti-cancer agents, anti-obesity agents or anti-diabetes agents. In certain embodiments, the fusion protein may be co-administered with insulin, preferably with an insulin glucose clamp. In certain embodiments, the fusion protein may be co-administered with an unfused amino acid enzyme targeting the same or another amino acid as the fusion protein. In certain embodiments, an arginase fusion protein may be administered with an asparaginase, e.g., asparaginase in its monomeric form as described above, either unfused or also in the form of an insulin fusion.

The present invention allows an improved insulin-mediated transcytosis and endocytosis of amino acid degrading enzymes by providing a fusion protein between the insulin and the enzyme instead of requiring the chance of entrapping a nearby enzyme molecule. Of the highest interest is a fusion protein between arginase and insulin, but other arginine degrading enzymes as well as some other enzymes degrading other amino acids, may be fused with insulin to increase their anti-tumor efficacy.

In addition to the use of the fusion protein of insulin and arginase as an anti-tumor medication, the same fusion protein can be used to treat obesity, particularly if obesity is concurrent with diabetes already being treated by insulin. Bringing arginase enzyme into fat cells that are a prime target for insulin, will inhibit fat cell growth and proliferation and possibly even cause some of them to die depending on the level of intracellular arginine depletion.

### Experimental Observations and Conclusions Therefrom

The inventor's research on systemic depletion of arginine and asparagine as anti-cancer treatments conducted on healthy experimental dogs and several dogs with cancer starting in 1995 and still in progress has provided strong evidence of the role for insulin in increasing the effectiveness of these treatments that rely on enzymatic degradation of the targeted amino acids.

In the first stages of this project, extracorporeal removal of targeted amino acids was carried out by selective dialysis. Using modified dialysis equipment, the blood was dialyzed against a dialysate containing most known, low molecular weight water-soluble components (in total, 52+electrolytes) of blood plasma except the targeted amino acid. The efficacy of the process was verified by measuring all amino acids at the inlet and the outlet of the dialysis filter. Most of the essential amino acids were targeted in these experiments, one at a time, with arginine being of the main interest for its depletion efficacy being established against different tumor lines tested in vitro. Continuous dialysis of days in duration, however, failed to significantly lower plasma concentration of any of the essential amino acids despite near total washing out of the targeted amino acid by the filter. The targeted amino acid concentration at the outlet of the filter was below detection but at the inlet the concentration remained near normal. Blood flow was very high—up to 300 ml/min with dogs of about 30 kg body weight. The failure of this approach was correctly assigned to homeostatic controls of the essential amino acids that were estimated to result in as much as a 10% of total body protein loss per day.

In follow-up experimental studies the inventor turned to using an insulin/glucose clamp to inhibit protein breakdown and stimulate protein synthesis. While still using selective dialysis with the same parameters, the concentration of plasma arginine could now be reduced about ten-fold from about 100 to about 10 μM, as shown in FIG. 1. Dashed lines show plasma arginine concentration in 2 dogs without insulin/glucose clamp. Solid lines show plasma arginine in 6 dogs with insulin/glucose clamp.

However, sampling the lymphatic system showed no reduction of arginine concentration, which at about 200 μM was even higher than normal plasma concentration. The conclusion was clear—while dialysis could lower arginine concentration in the plasma, the molecular exchange between the blood and the interstitial fluid by diffusion and convective transports could not compensate for the influx of amino acids from protein turnover in the body, mostly from muscle proteins.

Thereafter, the inventor turned to using enzymatic degradation of the targeted amino acid. In a first approach, targeting arginine for removal, the inventor used partially purified liver extract rich in arginase. In pre-terminal experiments in healthy dogs, autologous liver extract was delivered by bolus infusions every 3 hours, during 18 hours in total. Without insulin/glucose clamp, plasma arginine level dropped to near zero and returned to normal level before the next bolus infusion 3 hours later, FIG. 2, curve A. With an insulin/glucose clamp plasma arginine was lowered to below detection and held there for the 18-hour duration of the experiment, FIG. 2, curve B despite the extract still delivered by bolus infusions.

After these pilot experiments, the inventor turned to continuous infusion of enzymatically active substances from different sources, including recombinant enzymes.

Rapid drops and increases of plasma arginine with bolus injections without insulin/glucose clamp were forgotten until years later and some further evidence of insulin effects in this anti-cancer therapeutic modality.

In all of over one hundred sessions on healthy, experimental dogs and those few with cancers, the inventor has noticed loss of albumin and moderate oedemas with deployment of insulin/glucose clamp. Loss of plasma albumin was attributed to protein turnover and oedemas to unwanted, but not limiting side-effects.

However, closer examination of the previously observed oscillations of arginine with bolus infusions of liver extract, provided a clue to a new, unexpected role of insulin in addition to its protein turnover regulation effects.

Liver arginase is a monomer of about 35 kDa molecular weight, right in the middle of molecular weight range that glomerular filtration can remove from plasma-up to about 70 kDa. Albumin molecular weight is 72 kDa and there is very little loss of it by diffusion into extravascular fluid or by glomerular filtration. However, insulin is known to increase permeability of capillary vessels, and hence maintaining insulin concentration at supraphysiologic concentration for prolonged periods will allow some extravasation of albumin that causes oedema. As stated, this was not limiting to the protocol and could be compensated for by using standard diuretic drugs.

However, the role of insulin was crucial in making arginine depletion by arginase an effective mechanism beyond vascular system. At 35 kDa, arginase is quickly eliminated by glomerular filtration. Use of insulin/glucose clamp causes an increase in capillary permeability sufficient to cause extravasation of arginase, thus protecting it from elimination by kidneys. It also delivers the enzyme to interstitial fluid where the real effects of arginine depletion on cancer must be present. Clearing the plasma of arginine is a poor surrogate of enzymatic anti-cancer effectiveness. In all current clinical trials as anti-cancer treatments, arginase or arginine deiminase are PEGylated. PEGylating these enzymes prevents their extravasation which explains lack of clinical successes despite arginine elimination from blood plasma.

The above observations provide a plausible explanation of the insulin/glucose role in extravasation of arginase.

Our in vitro work with canine cancer cell lines (Wells J W, Evans C H, Scott M C, Rütgen BC, O'Brien T D, Modiano J F, Cvetkovic G, Tepic S. Arginase treatment prevents the recovery of canine lymphoma and osteosarcoma cells resistant to the toxic effects of prolonged arginine deprivation. PLOS One. 2013;8 (1): e54464. doi: 10.1371/journal.pone.0054464. Epub 2013 Jan. 24. PMID: 23365669; PMCID: PMC3554772.) has suggested that exceptional effectiveness of arginase in rapid killing of cancer cells was partly due to arginase attachment to or entry into cancer cells. Depleting arginine in extracellular environment was needed but not sufficient. Selectivity for cancer cells vs. healthy cells may in part be due to increased rates of endocytosis displayed by cancer cells.

Cancer and rapidly proliferating healthy cells are known to have higher expression of insulin receptors. However, in contrast to cancer cells, healthy cells respond to depletion of arginine by exiting cell cycle where in the rest phase they can survive for up to 3 weeks. By contrast, lack of cycle control, the hallmark of all cancers, leads them into, in most cases, metabolic death.

The present invention aims at exploiting the previous findings by providing a fusion protein of insulin and arginase, in particular of human insulin and human liver arginase (Arginase-1). Using an appropriate linker for the fusion preserves the activity of both insulin and arginase.

In the in vivo work we have carried out so far, the average effective doses of insulin and arginase were close to stochiometric. However, should the further in vivo work with a fused protein demonstrate differential efficiency of fused vs single proteins, a combination of the fused molecule and regular insulin and/or regular arginase may be used.

### FIGURE LEGENDS

FIG. 1: Effect of arginase administration on the plasma arginine concentration in dogs. Dashed lines show plasma arginine concentration in 2 dogs without insulin/glucose clamp. Solid lines show plasma arginine concentration in 6 dogs with insulin/glucose clamp. Combined arginase and insulin/glucose clamp show a reduction about ten-fold from about 100 μM to about 10 μM.

FIG. 2: Administration of autologous partially purified liver extract rich in arginase by bolus infusions every 3 hours, during 18 hours in total without insulin/glucose clamp (curve A) and with insulin/glucose clamp (curve B). Without insulin/glucose clamp, plasma arginine level dropped to near zero and returned to normal level before the next bolus infusion 3 h later. With insulin/glucose clamp plasma arginine was lowered to below detection and held there for 18 h.

FIG. 3: Fusion protein with human liver arginase (ARG-1) as the first domain, including a histidine tag, and human proinsulin as the second domain connected by a flexible linker.

FIG. 4: Fusion protein with human liver arginase (ARG-1) as the first domain, including a histidine tag, and human insulin as the second domain connected by a flexible linker after disulfide crosslinking and cleavage of proinsulin C-peptide.

FIG. 5: Fusion protein with human liver arginase (ARG-1) as the first domain, including a histidine tag, and human proinsulin as the second domain connected by a flexible linker showing C-peptide cleavage sites by PC 1/3 and cpE enzymes.

FIG. 6: Fusion protein with human liver arginase (ARG-1) as the first domain, including a histidine tag, and a Single Chain Insulin analog (SCI-57) with a permanent hexapeptide linker (C-Linker) between B and A chains, including 4 favorable amino acid substitutions (at B10, B28, B29, and As).

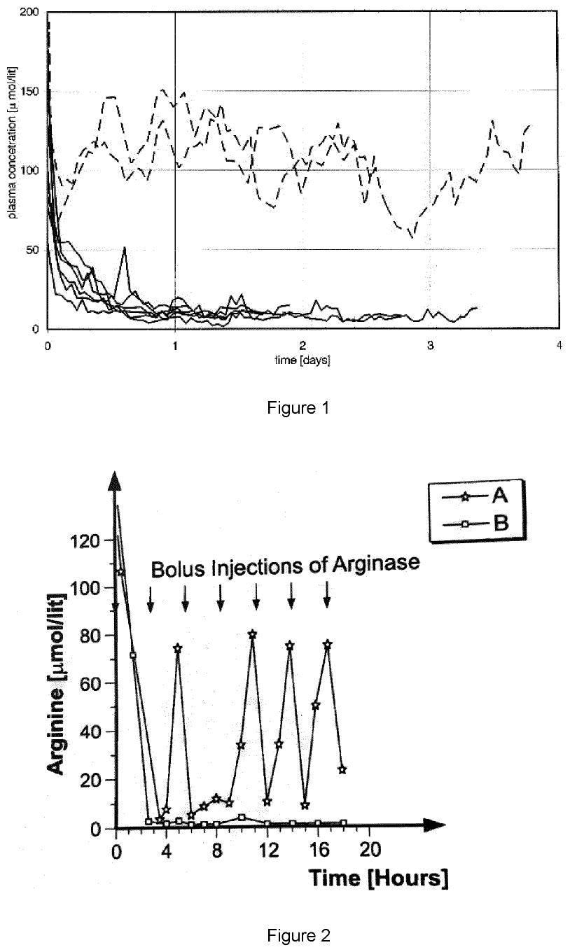

### EXAMPLE 1

A fusion protein comprising as N-terminal domain human liver arginase (UniProtKB, Arginase-1, P05089) and as C-terminal domain human proinsulin is provided. The human proinsulin is fused to the arginase with its N-terminus which is the start of the B-chain (UniProtKB, human insulin, P01308).

For this purpose, a suitable host cell, e.g., a prokaryotic cell such as E. coli, or a eukaryotic cell such as a yeast, insect, or mammalian cell, is transfected with a nucleic acid molecule encoding the fusion protein in operative linkage with an expression control sequence adapted for the respective host cell. The host cell is cultured under suitable conditions for expressing the fusion protein. The fusion protein is purified from the cell or from the culture medium according to known techniques.

After recombinant production crosslinking of B and A chains is effected by disulfide bonds and the C-peptide is removed enzymatically.

The sequence below of ARG-1 with a histidine tag added for affinity purification with total length of 331 amino acids the inventor has used to produce in E. coli a highly active human liver arginase:

(SEQ ID NO. 1)
MGHHHHHHGSSAKSRTIGIIGAPFSKGQPRGGVEEGPTVLRKAGLLEKL
KEQECDVKDYGDLPFADIPNDSPFQIVKNPRSVGKASEQLAGKVAEVKK
NGRISLVLGGDHSLAIGSISGHARVHPDLGVIWVDAHTDINTPLTTTSG
NLHGQPVSFLLKELKGKIPDVPGFSWVTPCISAKDIVYIGLRDVDPGEH
YILKTLGIKYFSMTEVDRLGIGKVMEETLSYLLGRKKRPIHLSFDVDGL
DPSFTPATGTPVVGGLTYREGLYITEEIYKTGLLSGLDIMEVNPSLGKT
PEEVTRTVNTAVAITLACFGLAREGNHKPIDYLNPPK

The proinsulin sequence:

(SEQ ID NO. 2)
FVNQHLCGSHLVEALYLVCGERGFFYTPKTRREAEDLQVGQVELGGGP
GAGSLQPLALEGSLQKRGIVEQCCTSICSLYQLENYCN

of human insulin is 86 amino acids long.

The B-chain sequence:

(SEQ ID NO. 3)
FVNQHLCGSHLVEALYLVCGERGFFYTPKT

at the start of proinsulin sequence is 30 amino acids long.

C-peptide:

(SEQ ID NO. 4)
RREAEDLQVGQVELGGGPGAGSLQPLALEGSLQKR

(underlined in the proinsulin sequence above) is 35 amino acids long and is enzymatically removed after cross-linking of the B and A-chains.

A-chain sequence:

(SEQ ID NO. 5)
GIVEQCCTSICSLYQLENYCN

is 21 amino acids long.

Disulfide bonds link cysteine residues at the 7^{th} position in both B and A chains, as well as cysteine residues at the 19^{th} position in the B-chain and the 20^{th} position in the A-chain. There is also an additional disulfide bond within the A-chain between cysteine residues at positions 6 and 11.

The first 3 (FVN) and the last 4 (TPKT) amino acids of the B-chain are not known to interact with either the A-chain or the insulin receptor. By contrast, only the last amino acid (N) in the A-chain is not known to participate in any interactions.

A linker X may be inserted between the C-terminus of arginase (K) and the N-terminus (F) of the B-chain of proinsulin. With only 3 amino acids of the B-chain at N-terminus not known to participate in any interactions, a flexible linker of GS-type is the first choice. A linker Y, e.g., a short GS linker, may be incorporated at the N-terminus of the arginase(S) to link a purification tag, e.g., a His-Tag to the arginase.

One of the most commonly used GS-type linkers is (G₄S)ₙ. The length of the linker is subject to its expected function—in this case to increase spatial separation of arginase and insulin and thus preserve their independent activities. In certain embodiments, a (G₄S)₁₀ (SEQ ID NO. 6) is used.

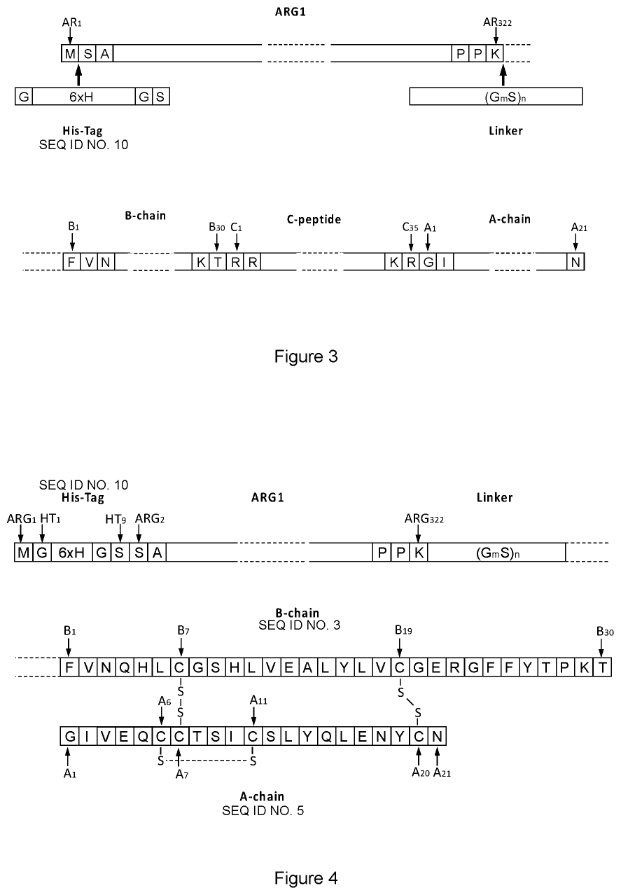

In a specific embodiment, the amino acid sequence of a fusion protein comprising a His purification tag, human arginase I and proinsulin is as follows (FIG. 3):

(SEQ ID NO. 7)
MGHHHHHH*Y*SAKSRTIGIIGAPFSKGQPRGGVEEGPTVLRKAGLLEK
LKEQECDVKDYGDLPFADIPNDSPFQIVKNPRSVGKASEQLAGKVAEVK
KNGRISLVLGGDHSLAIGSISGHARVHPDLGVIWVDAHTDINTPLTTTS
GNLHGQPVSFLLKELKGKIPDVPGFSWVTPCISAKDIVYIGLRDVDPGE
HYILKTLGIKYFSMTEVDRLGIGKVMEETLSYLLGRKKRPIHLSFDVDG
LDPSFTPATGTPVVGGLTYREGLYITEEIYKTGLLSGLDIMEVNPSLGK
TPEEVTRTVNTAVAITLACFGLAREGNHKPIDYLNPPK*X*FVNQHLCG
SHLVEALYLVCGERGFFYTPKTRREAEDLQVGQVELGGGPGAGSLQPLA
LEGSLQKRGIVEQCCTSICSLYQLENYCN,

wherein Y is a linker, particularly a GS linker, and X is a linker, particularly a (GGGGS) n linker, wherein n is from 1 to 10.

After removal of the C-peptide the fusion protein consists of two polypeptides, wherein polypeptide (i) consists of the His-tag, the arginase domain, the linker and the insulin B peptide, and polypeptide (ii) consists of the insulin A-chain, and wherein polypeptides (i) and (ii) are cross-linked by disulfide bonds, as follows (FIG. 4):

Polypeptide (i):

(SEQ ID NO. 8)
MGHHHHHH*Y*SAKSRTIGIIGAPFSKGQPRGGVEEGPTVLRKAGLLEK
LKEQECDVKDYGDLPFADIPNDSPFQIVKNPRSVGKASEQLAGKVAEVK
KNGRISLVLGGDHSLAIGSISGHARVHPDLGVIWVDAHTDINTPLTTTS
GNLHGQPVSFLLKELKGKIPDVPGFSWVTPCISAKDIVYIGLRDVDPGE
HYILKTLGIKYFSMTEVDRLGIGKVMEETLSYLLGRKKRPIHLSFDVDG
LDPSFTPATGTPVVGGLTYREGLYITEEIYKTGLLSGLDIMEVNPSLGK
TPEEVTRTVNTAVAITLACFGLAREGNHKPIDYLNPPK*X*FVNQHLCG
SHLVEALYLVCGERGFFYTPKT,

wherein Y is a linker, particularly a GS linker, and X is a linker, particularly a (GGGGS) n linker, wherein n is from 1 to 10.

Polypeptide (ii):

(SEQ ID NO. 5)
GIVEQCCTSICSLYQLENYCN

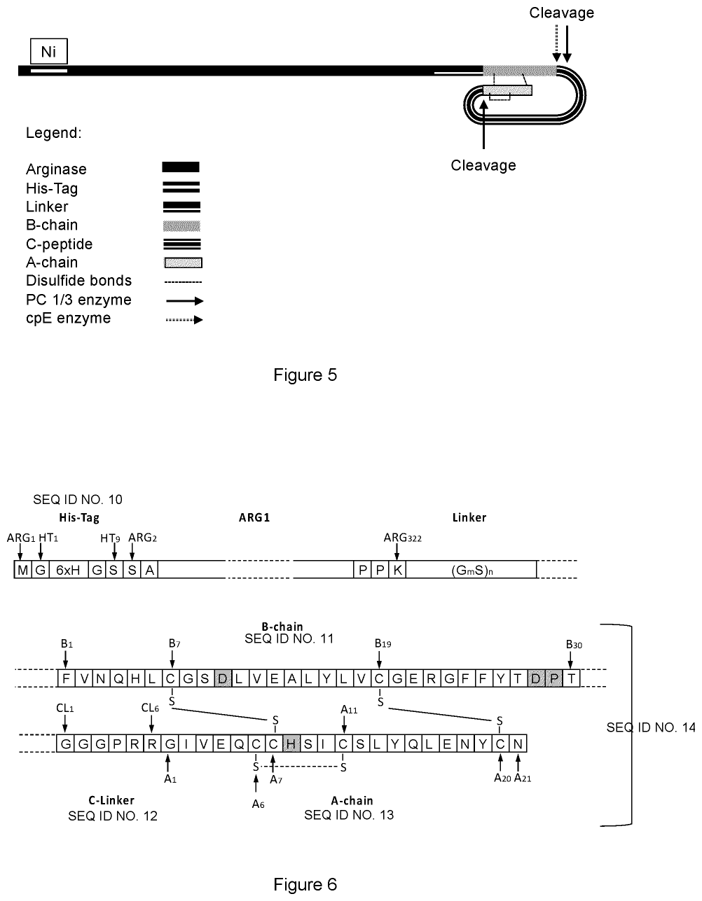

Disulfide cross-linking of the insulin chains and the enzymatic removal of the C-peptide is preferably carried out with the fusion protein bound to a purification material, e.g. a purification column or filter, by its affinity tag. In certain embodiments, the purification material is an affinity filter or column, e.g., a Ni filter or column to which the fusion protein is bound by its His-Tag (FIG. 5).

The length of the fusion protein with a G₄S linker (SEQ ID NO. 9) is 331+5+30+21=387 residues and the molecular weight is approximately 42.0 kDa.

With a (G₄S)₁₀ (SEQ ID NO. 6) linker the length is 432 residues, and the molecular weight is approximately 45.6 kDa.

Assuming that the increase in permeability of the vascular system due to insulin is a result of its mass transport through endothelial cells by transcytosis, which probably also causes extravasation of arginase of 35.8 kDa (with His-Tag), it is reasonable to expect a similar, but due to “tag-along” effect of insulin on arginase, potentially even higher, rate of transcytosis of the fusion protein of about 42 to 46 kDa.

The same is true for entry into target cells by endocytosis, specifically cancerous cells known to overexpress insulin receptors. In many in vivo experiments we have performed using arginase and insulin there were no major ill-effects on healthy cells-only transiently reduced proliferation rate.

### EXAMPLE 2

A further embodiment of the invention is a fusion protein of insulin with human Arginase-2, found predominantly in the kidneys and several other tissues but not in the liver (alternative names: Arginase II, Kidney-type arginase, non-hepatic arginase). Arginase-2 (UniProt P78540) is 354 residues long and has molecular weight of 38,578 Da. Using the same His-Tag (GHHHHHHGS) (SEQ ID NO. 10) at the N-terminus as for Arginase-1, this protein would have 363 residues and molecular weight of approximately 39.7 kDa. With a (G4S) n linker as described above, the fusion protein of human Arginase-2 and insulin would have molecular weight of 46 to 50 kDa.

### EXAMPLE 3

A further embodiment of the invention is a fusion protein of insulin with a human cobalt-substituted Arginase I or Arginase II. This fusion protein may be produced as described in US 20121/0189371, supra, comprising fermentation of E. coli cells expressing the fusion protein to produce the fusion protein and substitution of cobalt for manganese in the recombinant protein to provide a Co-substituted fusion protein which may be further purified.

### EXAMPLE 4

A further embodiment of the present invention is the fusion protein of human Arginase-1 and SCI-57 (FIG. 6). In addition to the permanent hexapeptide C-linker GGGPRR (SEQ ID NO. 12) replacing C-peptide of native proinsulin, the B-chain chain (SEQ ID NO. 11) and the A-chain (SEQ ID NO. 13) of SCI-57 comprise further modifications to the human insulin, namely 4 substitutions: ThrA8 to His, HisB10 to Asp, ProB28 to Asp, and LysB29 to Pro, shown in gray on FIG. 6. SCI-57 (SEQ ID NO. 14) is an ultra-stable single chain insulin of high binding ability to the insulin receptor. Of particular interest to the present invention is the use of a permanent peptide linker in SCI-57, allowing for a single step production of the fusion protein with monomeric arginase.

### EXAMPLE 5

The fusion protein of the invention is administered to a patient by infusion. For systemic, deep depletion called for in anti-tumor therapy, delivery of the fusion protein is preferably performed with co-infusion of glucose and optionally additional measures such as co-infusion of a nitric oxide donor (e.g., SNP) and/or a vasopressin (e.g., arginine-vasopressin) to compensate for side effects of low arginine.

A lower dose would be needed for treating obesity and/or type-2 diabetes with the dosage close to the standard use of insulin alone.

The sequences listed as SEQ ID NO. 1 to SEQ ID NO. 14 according to the present invention are defined as follows:

(ARG-1 with histidine tag)
SEQ ID NO. 1
Met Gly His His His His His His Gly Ser Ser Ala Lys Ser Arg Thr
Ile Gly Ile Ile Gly Ala Pro Phe Ser Lys Gly Gln Pro Arg Gly Gly
Val Glu Glu Gly Pro Thr Val Leu Arg Lys Ala Gly Leu Leu Glu Lys
Leu Lys Glu GIn Glu Cys Asp Val Lys Asp Tyr Gly Asp Leu Pro Phe
Ala Asp Ile Pro Asn Asp Ser Pro Phe Gln Ile Val Lys Asn Pro Arg
Ser Val Gly Lys Ala Ser Glu Gln Leu Ala Gly Lys Val Ala Glu Val
Lys Lys Asn Gly Arg Ile Ser Leu Val Leu Gly Gly Asp His Ser Leu
Ala Ile Gly Ser Ile Ser Gly His Ala Arg Val His Pro Asp Leu Gly
Val Ile Trp Val Asp Ala His Thr Asp Ile Asn Thr Pro Leu Thr Thr
Thr Ser Gly Asn Leu His Gly Gln Pro Val Ser Phe Leu Leu Lys Glu
Leu Lys Gly Lys Ile Pro Asp Val Pro Gly Phe Ser Trp Val Thr Pro
Cys Ile Ser Ala Lys Asp Ile Val Tyr Ile Gly Leu Arg Asp Val Asp
Pro Gly Glu His Tyr Ile Leu Lys Thr Leu Gly Ile Lys Tyr Phe Ser
Met Thr Glu Val Asp Arg Leu Gly Ile Gly Lys Val Met Glu Glu Thr
Leu Ser Tyr Leu Leu Gly Arg Lys Lys Arg Pro Ile His Leu Ser Phe
Asp Val Asp Gly Leu Asp Pro Ser Phe Thr Pro Ala Thr Gly Thr Pro
Val Val Gly Gly Leu Thr Tyr Arg Glu Gly Leu Tyr Ile Thr Glu Glu
Ile Tyr Lys Thr Gly Leu Leu Ser Gly Leu Asp Ile Met Glu Val Asn
Pro Ser Leu Gly Lys Thr Pro Glu Glu Val Thr Arg Thr Val Asn Thr
Ala Val Ala Ile Thr Leu Ala Cys Phe Gly Leu Ala Arg Glu Gly Asn
His Lys Pro Ile Asp Tyr Leu Asn Pro Pro Lys
(human protein)
SEQ ID NO. 2
Phe Val Asn Gln His Leu Cys Gly Ser His Leu Val Glu Ala Leu Tyr
Leu Val Cys Gly Glu Arg Gly Phe Phe Tyr Thr Pro Lys Thr Arg Arg
Glu Ala Glu Asp Leu Gin Val Gly Gln Val Glu Leu Gly Gly Gly Pro
Gly Ala Gly Ser Leu Gln Pro Leu Ala Leu Glu Gly Ser Leu Gln Lys
Arg Gly Ile Val Glu GIn Cys Cys Thr Ser Ile Cys Ser Leu Tyr Gln
Leu Glu Asn Tyr Cys Asn
(B-chain)
SEQ ID NO. 3
Phe Val Asn Gln His Leu Cys Gly Ser His Leu Val Glu Ala Leu Tyr
Leu Val Cys Gly Glu Arg Gly Phe Phe Tyr Thr Pro Lys Thr
(C-peptide)
SEQ ID NO. 4
Arg Arg Glu Ala Glu Asp Leu Gln Val Gly Gln Val Glu Leu Gly Gly
Gly Pro Gly Ala Gly Ser Leu Gln Pro Leu Ala Leu Glu Gly Ser Leu
Gln Lys Arg
(A-chain/polypeptide (ii))
SEQ ID NO. 5
Gly Ile Val Glu GIn Cys Cys Thr Ser Ile Cys Ser Leu Tyr Gln Leu
Glu Asn Tyr Cys Asn
((G4S)10 linker)
SEQ ID NO. 6
Gly Gly Gly Gly Ser Gly Gly Gly Gly Ser Gly Gly Gly Gly Ser Gly
Gly Gly Gly Ser Gly Gly Gly Gly Ser Gly Gly Gly Gly Ser Gly Gly
Gly Gly Ser Gly Gly Gly Gly Ser Gly Gly Gly Gly Ser Gly Gly Gly
Gly Ser
(Artificial Sequence)
SEQ ID NO. 7
Xaa on position 9 = linker, particularly a GS linker
Xaa on position 331 = linker, particularly a (GGGGS)n linker, wherein n is from
1 to 10
Met Gly His His His His His His Xaa Ser Ala Lys Ser Arg Thr Ile
Gly Ile Ile Gly Ala Pro Phe Ser Lys Gly Gln Pro Arg Gly Gly Val
Glu Glu Gly Pro Thr Val Leu Arg Lys Ala Gly Leu Leu Glu Lys Leu
Lys Glu GIn Glu Cys Asp Val Lys Asp Tyr Gly Asp Leu Pro Phe Ala
Asp Ile Pro Asn Asp Ser Pro Phe Gln Ile Val Lys Asn Pro Arg Ser
Val Gly Lys Ala Ser Glu GIn Leu Ala Gly Lys Val Ala Glu Val Lys
Lys Asn Gly Arg Ile Ser Leu Val Leu Gly Gly Asp His Ser Leu Ala
Ile Gly Ser Ile Ser Gly His Ala Arg Val His Pro Asp Leu Gly Val
Ile Trp Val Asp Ala His Thr Asp Ile Asn Thr Pro Leu Thr Thr Thr
Ser Gly Asn Leu His Gly Gln Pro Val Ser Phe Leu Leu Lys Glu Leu
Lys Gly Lys Ile Pro Asp Val Pro Gly Phe Ser Trp Val Thr Pro Cys
Ile Ser Ala Lys Asp Ile Val Tyr Ile Gly Leu Arg Asp Val Asp Pro
Gly Glu His Tyr Ile Leu Lys Thr Leu Gly Ile Lys Tyr Phe Ser Met
Thr Glu Val Asp Arg Leu Gly Ile Gly Lys Val Met Glu Glu Thr Leu
Ser Tyr Leu Leu Gly Arg Lys Lys Arg Pro Ile His Leu Ser Phe Asp
Val Asp Gly Leu Asp Pro Ser Phe Thr Pro Ala Thr Gly Thr Pro Val
Val Gly Gly Leu Thr Tyr Arg Glu Gly Leu Tyr Ile Thr Glu Glu Ile
Tyr Lys Thr Gly Leu Leu Ser Gly Leu Asp Ile Met Glu Val Asn Pro
Ser Leu Gly Lys Thr Pro Glu Glu Val Thr Arg Thr Val Asn Thr Ala
Val Ala Ile Thr Leu Ala Cys Phe Gly Leu Ala Arg Glu Gly Asn His
Lys Pro Ile Asp Tyr Leu Asn Pro Pro Lys Xaa Phe Val Asn Gln His
Leu Cys Gly Ser His Leu Val Glu Ala Leu Tyr Leu Val Cys Gly Glu
Arg Gly Phe Phe Tyr Thr Pro Lys Thr Arg Arg Glu Ala Glu Asp Leu
Gln Val Gly Gln Val Glu Leu Gly Gly Gly Pro Gly Ala Gly Ser Leu
Gln Pro Leu Ala Leu Glu Gly Ser Leu Gln Lys Arg Gly Ile Val Glu
Gln Cys Cys Thr Ser Ile Cys Ser Leu Tyr Gln Leu Glu Asn Tyr Cys
Asn
(polypeptide (i))
SEQ ID NO. 8
Xaa on position 9 = linker, particularly a GS linker
Xaa on position 331 = linker, particularly a (GGGGS)n linker, wherein n is from 1
to 10
Met Gly His His His His His His Xaa Ser Ala Lys Ser Arg Thr Ile
Gly Ile Ile Gly Ala Pro Phe Ser Lys Gly Gln Pro Arg Gly Gly Val
Glu Glu Gly Pro Thr Val Leu Arg Lys Ala Gly Leu Leu Glu Lys Leu
Lys Glu GIn Glu Cys Asp Val Lys Asp Tyr Gly Asp Leu Pro Phe Ala
Asp Ile Pro Asn Asp Ser Pro Phe Gln Ile Val Lys Asn Pro Arg Ser
Val Gly Lys Ala Ser Glu GIn Leu Ala Gly Lys Val Ala Glu Val Lys
Lys Asn Gly Arg Ile Ser Leu Val Leu Gly Gly Asp His Ser Leu Ala
Ile Gly Ser Ile Ser Gly His Ala Arg Val His Pro Asp Leu Gly Val
Ile Trp Val Asp Ala His Thr Asp Ile Asn Thr Pro Leu Thr Thr Thr
Ser Gly Asn Leu His Gly Gln Pro Val Ser Phe Leu Leu Lys Glu Leu
Lys Gly Lys Ile Pro Asp Val Pro Gly Phe Ser Trp Val Thr Pro Cys
Ile Ser Ala Lys Asp Ile Val Tyr Ile Gly Leu Arg Asp Val Asp Pro
Gly Glu His Tyr Ile Leu Lys Thr Leu Gly Ile Lys Tyr Phe Ser Met
Thr Glu Val Asp Arg Leu Gly Ile Gly Lys Val Met Glu Glu Thr Leu
Ser Tyr Leu Leu Gly Arg Lys Lys Arg Pro Ile His Leu Ser Phe Asp
Val Asp Gly Leu Asp Pro Ser Phe Thr Pro Ala Thr Gly Thr Pro Val
Val Gly Gly Leu Thr Tyr Arg Glu Gly Leu Tyr Ile Thr Glu Glu Ile
Tyr Lys Thr Gly Leu Leu Ser Gly Leu Asp Ile Met Glu Val Asn Pro
Ser Leu Gly Lys Thr Pro Glu Glu Val Thr Arg Thr Val Asn Thr Ala
Val Ala Ile Thr Leu Ala Cys Phe Gly Leu Ala Arg Glu Gly Asn His
Lys Pro Ile Asp Tyr Leu Asn Pro Pro Lys Xaa Phe Val Asn Gln His
Leu Cys Gly Ser His Leu Val Glu Ala Leu Tyr Leu Val Cys Gly Glu
Arg Gly Phe Phe Tyr Thr Pro Lys Thr
(G4S linker)
SEQ ID NO. 9
Gly Gly Gly Gly Ser
(His-Tag)
SEQ ID NO. 10
Gly His His His His His His Gly Ser
(B-chain)
SEQ ID NO. 11
Phe Val Asn Gln His Leu Cys Gly Ser Asp Leu Val Glu Ala Leu Tyr
Leu Val Cys Gly Glu Arg Gly Phe Phe Tyr Thr Asp Pro Thr
(C-linker)
SEQ ID NO. 12
Gly Gly Gly Pro Arg Arg
(A-chain)
SEQ ID NO. 13
Gly Ile Val Glu Gln Cys Cys His Ser Ile Cys Ser Leu Tyr Gln Leu
Glu Asn Tyr Cys Asn
(SCI-57)
SEQ ID NO. 14
Phe Val Asn Gln His Leu Cys Gly Ser Asp Leu Val Glu Ala Leu Tyr
Leu Val Cys Gly Glu Arg Gly Phe Phe Tyr Thr Asp Pro Thr Gly Gly
Gly Pro Arg Arg Gly Ile Val Glu Gln Cys Cys His Ser Ile Cys Ser
Leu Tyr Gln Leu Glu Asn Tyr Cys Asn

1. A fusion protein comprising a first domain and a second domain wherein the first domain comprises an amino acid degrading enzyme and the second domain comprises an insulin.

2. The fusion protein of claim 1 wherein the first domain (enzyme) is located N-terminally to the second domain (insulin).

3. The fusion protein of claim 1, which is a genetic fusion.

4. The fusion protein of claim 1, wherein the amino acid degrading enzyme is an arginine degrading enzyme, e.g., an arginine deiminase (ADI) or an arginase.

5. The fusion protein of claim 1, wherein the amino acid degrading enzyme is a human arginase such as human liver arginase (human Arginase-1), or human kidney arginase (human Arginase-2).

6. The fusion protein of claim 1, wherein the amino acid degrading enzyme is a monomer protein, e.g., a monomeric arginase.

7. The fusion protein of claim 1, wherein the insulin is a human insulin or an insulin analogue including a single-chain insulin.

8. The fusion protein of claim 1, wherein the first domain and the second domain are connected to each other by a linker.

9. The fusion protein of claim 8, wherein the linker is a flexible linker, e.g., a linker composed of the amino acids G and S, e.g., a (GmS) n linker wherein m is from 1-5 and n is from 1-10, a rigid linker, or a cleavable linker.

10. A nucleic acid molecule encoding the fusion protein of claim 1.

11. A host cell transfected with the nucleic acid molecule of claim 10.

12. A method of producing the fusion protein of claim 1 by cultivating a host cell transfected with a nucleic acid molecule encoding the fusion protein of claim 1 and obtaining the fusion protein from the host cell or from the culture medium.

13. The fusion protein of claim 1 in combination with a carrier suitable for use in medicine.

14. A method for treating cancer or preventing or treating a metabolic disorder comprising administering a fusion protein of claim 1 to a patient in need of such treatment.

15. The method of claim 14, wherein the administration of the fusion protein of claim 1 is accompanied by co-administration of glucose and optionally accompanied by measures to compensate side-effects of arginine depletion.

16. The method for treating cancer or preventing or treating a metabolic disorder according to claim 14, wherein said metabolic disorder is obesity or diabetes.

17. The method for treating cancer or preventing or treating a metabolic disorder according to claim 16, wherein said metabolic disorder is type 2 diabetes.
